(19) United States
(12) Patent Application Publication
(10) Pub. No.: US 2024/0390097 A1
(43) Pub. Date: Nov. 28, 2024

(54) BOX INTENDED TO RECEIVE WASTE, IN PARTICULAR MEDICAL WASTE

(71) Applicant: EUROPE METAL CONCEPT (EMC), GRAVESON (FR)

(72) Inventor: Louis PIAZZA, EYRAGUES (FR)

(21) Appl. No.: 18/697,310

(22) PCT Filed: Sep. 22, 2022
(86) PCT No.: PCT/FR2022/051781

§ 371 Date: Apr. 15, 2024

(30) Foreign Application Priority Data
Sep. 30, 2021 (FR) FR2110307

(51) Int. Cl. A61B 50/39; A61L 11/00; B65F 1/08; B65F 1/16

(52) U.S. Cl. CPC A61B 50/39; A61L 11/00; B65F 1/08; B65F 1/16

(57) ABSTRACT

A box including: a receptacle; a plate fastened on the receptacle and including an aperture for the introduction of wastes; a cartridge containing a waste disinfection fluid, the cartridge being mounted in the receptacle and including a casing and an actuator movable relative to the casing between an inactive position and an active position in which fluid can be expelled through an expulsion hole; a cover which is mounted on the plate and which includes means for activating the actuator of the cartridge. When the cover is in an open position, the actuator is in an inactive position, and the switch of the cover into the closed position causes the switch of the actuator into the active position by the action of the activation means.

### CROSS REFERENCE TO RELATED APPLICATIONS

This application is a National Stage of PCT Application No. PCT/FR2022/051781 filed on Sep. 22, 2022, which claims priority to French Patent Application No. 21/10307 filed on Sep. 30, 2021, the contents each of which are incorporated herein by reference thereto.

### TECHNICAL FIELD

The present disclosure relates to a box intended to receive wastes, in particular medical wastes.

### BACKGROUND

Such wastes may typically be derived from diagnostic or treatment activities in the fields of human and veterinary medicine, and may have an infectious risk because they contain micro-organisms which could cause diseases in humans or in other living organisms. They may consist of dressings, syringes, bistouris, tubings, etc.

These wastes, which may be called DASRI (wastes of care activities with infectious risks), are subject to a particular regulation. In particular, the storage and the transport of these wastes must be carried out in suitable boxes; then these wastes as well as the box that contains them are eliminated by incineration or by burial, in a branch different from that dedicated to household wastes.

On the one hand, the elimination of these wastes requires relatively heavy and expensive processes, in order to guarantee that they can no longer present any infectious risk once eliminated. For example, the temperature of incineration of these wastes is significantly higher than that of household wastes.

On the other hand, the need to also eliminate the box, which also potentially has an infectious risk, generates a very large volume of elements that are discarded because they are unusable, which is obviously not desirable.

### BRIEF SUMMARY

The present disclosure aims to overcome the aforementioned drawbacks.

To this end, the present disclosure relates to a box intended to receive wastes, in particular medical wastes, comprising:
a receptacle including a peripheral wall, a bottom wall, and an opening opposite to the bottom wall;
a plate fastened to the receptacle, substantially covering the opening of the receptacle, and including at least one aperture for introducing wastes into the receptacle;
a cover mounted on the plate movably between an open position, in which the aperture of the plate is accessible, and a closed position, in which the cover seals said aperture;

By “the plate substantially covers the opening of the receptacle”, it should be understood that the plate completely covers said opening, with the exception of the area where the aperture for the introduction of wastes into the receptacle is located.

According to a general definition of the present disclosure, the box further includes a cartridge adapted to contain a waste disinfection fluid, the cartridge being mounted in the receptacle and including:
a casing;
a fluid expulsion hole;
an actuator movable relative to the casing between an inactive position in which the fluid cannot be expelled from the cartridge and an active position in which fluid can be expelled through the expulsion hole;

and the cover includes means for activating the actuator of the cartridge.

In addition, the box is configured so that, when the cover is in the open position, the actuator is in the inactive position, and so that the switch of the cover into the closed position causes the switch of the actuator into the active position by the action of the activation means.

Hence, the present disclosure allows disinfecting the wastes contained in the receptacle, and that being so automatically when the practitioner closes the cover, which corresponds to the protocol of use of the box once it is filled, before handling thereof by a specialized company.

The wastes then no longer have an infectious risk, and can be treated in the household wastes branch, that is to say according to less constraining and less expensive processes. Furthermore, once emptied of these disinfected wastes, the receptacle can be reused to form a new box, preferably by being cleaned beforehand and disinfected. Thus, it is possible to achieve a very significant reduction in the total volume of wastes.

In practice, the switch of the actuator into the active position may be performed at the very end of the closure movement of the cover. In practice, the configuration of the box enabling the activation means formed on the cover to act on the actuator may correspond to a configuration suited to this end of the cartridge and of the cover (in particular the structure of these two elements and their mutual arrangement), as described hereinafter.

According to a possible embodiment, the activation means comprise a lug projecting substantially orthogonally from the internal face of the cover. Furthermore, the plate includes an orifice which, when the cover is in the closed position, is located opposite the lug of the cover and the actuator of the cartridge, so that the lug can cooperate with the actuator to make it switch into its active position.

Alternatively, the cover could be devoid of any lug, and have a substantially planar lower face which cooperates with an actuator projecting above the plate.

According to different possibilities, the lug projects beneath the plate, or the actuator projects above the plate, or a contact face between the lug and the actuator upon cooperation thereof is located across the thickness of the plate.

In the closed position of the cover, the lug may be at least partially accommodated in the orifice of the plate. Preferably, the orifice of the plate has dimensions slightly larger than those of the lug, in order to ensure appropriate guidance and positioning of the lug, in particular with respect to the actuator.

According to a possible embodiment, the actuator includes an endpiece mounted at the upper portion of the cartridge, movable in vertical translation between an upper position, corresponding to the inactive position, and a lower position, corresponding to the active position, the endpiece being arranged opposite an orifice of the plate so as to be able to cooperate with the activation means formed on the cover.

The fluid expulsion hole may be formed in the endpiece, the endpiece thus forming a portion of an aerosol valve system.

The endpiece may be engaged at least partially into the orifice of the plate in the closed position of the cover. For example, the orifice of the plate has dimensions slightly larger than those of the endpiece, to ensure guidance of the latter.

Preferably, the orifice of the plate is distinct from the aperture through which the wastes can be introduced.

It could be provided for the endpiece to be located beneath the upper face of the plate in the inactive position. Such an arrangement allows preventing an untimely accidental actuation of the endpiece. Consequently, there could no longer be enough disinfectant fluid in the cartridge to effectively disinfect the wastes once the cover has been closed. In concrete terms, the upper face of the endpiece may be flush with the upper face of the plate, be located across the thickness of the plate, or be located beneath the lower face of the plate.

The cartridge may be mounted in the receptacle in a non-removable manner, the box including means for recharging the cartridge with fluid. By “non-removable”, it should be understood that the cartridge is permanently fastened in the receptacle and cannot be detached under normal conditions of use, of course unless a great force is implemented or tools are used.

Alternatively, the cartridge could be removably mounted in the receptacle. In this case, once empty, the cartridge could be replaced with a full cartridge.

According to a possible embodiment, the recharging means are formed on the cartridge and/or on the receptacle and are accessible from outside the box.

For example, the recharging means include a part which is mounted in a hole of the receptacle, opposite the cartridge, movably between an open position, enabling entry of the waste disinfection fluid into the cartridge, and a closed position ensuring sealing to said fluid between the inside and the outside of the container. The part may be biased towards its closed position by a spring. The switch into the open position may be obtained by the head of a fluid injection system which is sealingly inserted into said part, and which exerts a pressure on said part against the force exerted by the spring.

Alternatively, the recharging could be performed by the endpiece.

The cartridge may be disposed in the receptacle proximate to or against the peripheral wall of the receptacle. In particular, this allows leaving a large volume for receiving the wastes. With this arrangement, and in order not to interfere with the cartridge, provision may be made for the aperture for introducing the wastes to be substantially central on the plate. An advantage of disposing the cartridge against the peripheral wall of the receptacle—that is to say without a space between the cartridge and the peripheral wall of the receptacle—is to limit restricted spaces that are difficult to disinfect within the receptacle.

According to a possible embodiment, in the initial state, that is to say before mounting or during a step of mounting the different constituents of the box, the casing forms an open volume. In addition, the box includes at least one closure wall which, in the ready-to-use state, cooperates with the casing to form a closed volume adapted to contain the fluid. By “ready-to-use state”, it should be understood the state in which the box is supplied to the practitioner so that he/she can dispose the wastes therein. Hence, the box is mounted, in particular with the cartridge placed in the receptacle and the plate mounted on the receptacle. Having, in the ready-to-use state, a cartridge integrated into the container is very advantageous. Indeed, this considerably limits the interstices where infectious agents could enter, and where it would be difficult to neutralize them. Such an arrangement allows addressing a problem of molding the closed volume of the cartridge.

The closure wall may be formed at least partially by a wall of the receptacle. For example, the closure wall may be formed by a portion of the bottom wall and the peripheral wall of the receptacle, which may respectively form at least one portion of the bottom wall and of the peripheral wall of the casing of the cartridge. Alternatively, or complementarily, the casing may be made into several parts which are assembled, such as two half-shells. The closure wall may include or consist of one of these parts (for example of one of these parts and of a portion of a wall of the receptacle).

Alternatively, the closure wall could be a wall distinct from the casing and from the container, in the initial state. For example, the casing of the cartridge may be a cylinder having an upper end, the closure wall being a disk sealing this upper end.

According to one embodiment, the casing is a part distinct from the receptacle, in the initial state, which includes a lower opening and a lateral opening, said casing being sealingly fastened against the internal face of the receptacle, so that the bottom wall of the receptacle forms the bottom of the cartridge and the peripheral wall of the receptacle closes the lateral opening of the casing.

It could be provided for the area of the bottom wall of the receptacle that forms the bottom of the cartridge and for the area of the peripheral wall of the receptacle that closes the lateral opening of the casing to form a local extra thickness, towards the inside of the receptacle, relative to the adjacent areas of the bottom wall and of the peripheral wall of the receptacle, respectively, enabling guidance and nesting of the casing against the receptacle.

According to another embodiment, the box includes at least two initially distinct portions, each portion constituting a receptacle portion and a casing portion and preferably being made in one-piece by molding, said two portions being sealingly assembled to form the receptacle and the cartridge integrated into the receptacle. These two portions may substantially consist of halves, on both sides of a vertical midplane of the box. The assembly may be performed by heat-welding.

The area of the bottom wall of the receptacle that forms the bottom of the cartridge may have a portion having a shape cambered towards the inside of the receptacle, promoting the appropriate expulsion of the disinfection fluid.

According to a second aspect, the present disclosure relates to a method for recycling a box as described before, the cover being in the closed position. The method includes the following steps:
detaching the assembly comprising the plate and the cover from the receptacle;
emptying the wastes contained in the receptacle;
cleaning and disinfecting the receptacle;
recharging the cartridge with disinfection fluid, or replacing the cartridge with a cartridge full of disinfection fluid;
mounting the assembly comprising the plate and the cover again on the receptacle.

The wastes may be emptied like household wastes since they have been disinfected upon closure of the cover.

Upon completion of this process, and once the cover is placed in the open position, the box can be used again by a practitioner.

The box is generally designed so that a practitioner cannot detach the plate from the receptacle or open the cover once it has been closed. These operations can be carried out only by a specialized company, typically by means of suitable machines.

### BRIEF DESCRIPTION OF THE DRAWINGS

Several possible embodiments of the present disclosure are now described, as non-limiting examples, with reference to the appended figures:

FIG. 1 is a perspective view of a box according to an embodiment of the present disclosure, the cover being in the closed position;

FIG. 2 is a perspective view of the box of FIG. 1, the cover being in the open position;

FIG. 3 is a detail view of FIG. 3, showing locking of the cover on the plate;

FIG. 4 is a perspective view of the box of FIG. 1, showing a cartridge inside the receptacle;

FIG. 5 is a schematic vertical cross-sectional view of the plate and of the cover of the box;

FIG. 6 is a schematic and partial vertical longitudinal section view of the box;

FIG. 7 is a top view of the open box, showing the inside of the receptacle in the absence of a cartridge, according to one embodiment;

FIG. 8 is a detail view of the bottom wall of the receptacle of FIG. 7, according to a longitudinal vertical section;

FIG. 9 is a perspective view of a cartridge according to one embodiment;

FIG. 10 is a perspective view, according to another angle of view, of the cartridge of FIG. 9;

FIG. 11 is a top view of the open box, showing the inside of the receptacle of FIG. 6 equipped with the cartridge of FIGS. 9 and 10;

FIG. 12 illustrates a step of manufacturing the receptacle of a box according to another embodiment;

FIG. 13 is a detail view in vertical longitudinal section illustrating means for recharging the cartridge with fluid, in the inactive state;

FIG. 14 is a view similar to FIG. 13, the means for recharging the cartridge with fluid being in the active state, during recharging.

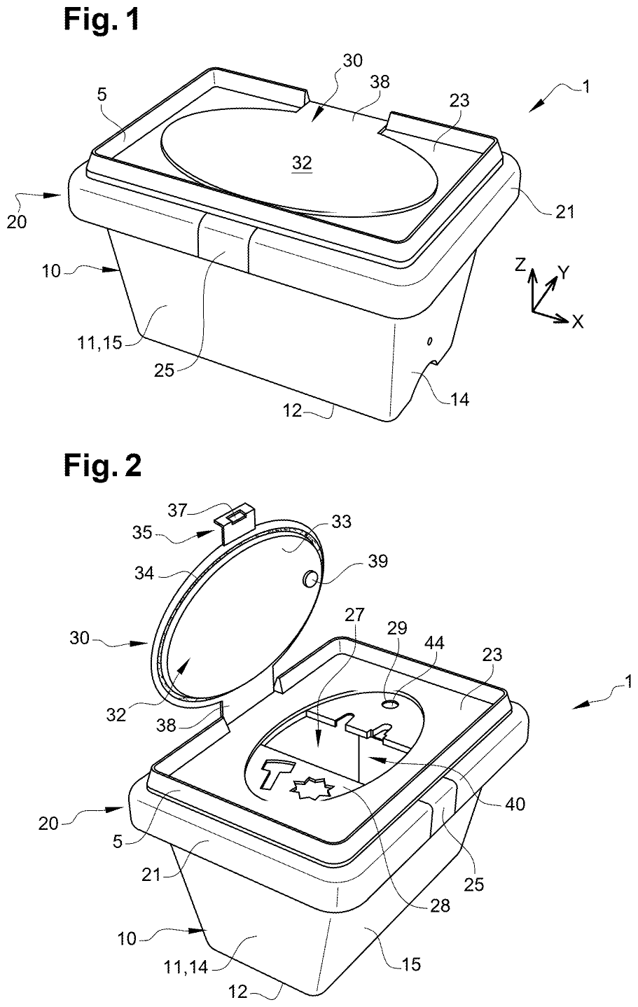

FIG. 1 represents a box 1 according to an embodiment of the present disclosure, which is typically intended to receive medical wastes having an infectious risk.

### DETAILED DESCRIPTION

The box 1 includes a receptacle 10 which includes a peripheral wall 11, a bottom wall 12, and an opening 13 opposite to the bottom wall 12. The receptacle 10 may be substantially parallelepipedal, possibly with a peripheral wall 11 which flares from the bottom wall 12 in the direction of the opening 13. Thus, according to this embodiment, the bottom wall 12 is substantially rectangular and generally planar, and the peripheral wall 11 includes two small panels 14 opposite one another and two large panels 15 opposite one another. The receptacle 10 may be made by molding a plastic material.

The box 1 is described when the bottom wall 12 rests on a horizontal surface, that is to say in the position illustrated in FIG. 1 in particular. The direction Z is defined as the direction orthogonal to the bottom wall 12, and therefore vertical in the described position, the longitudinal direction X as the direction orthogonal to Z and to the small panels 14, and the transverse direction Y as the direction orthogonal to X and to Z. This position is that in which the box 1 is used by a practitioner. Nonetheless, it should be understood that the box may take on other orientations in space, in particular during transport thereof.

The terms “above”, “high”, “low”, “upper”, “lower” and the like are defined with reference to the direction Z

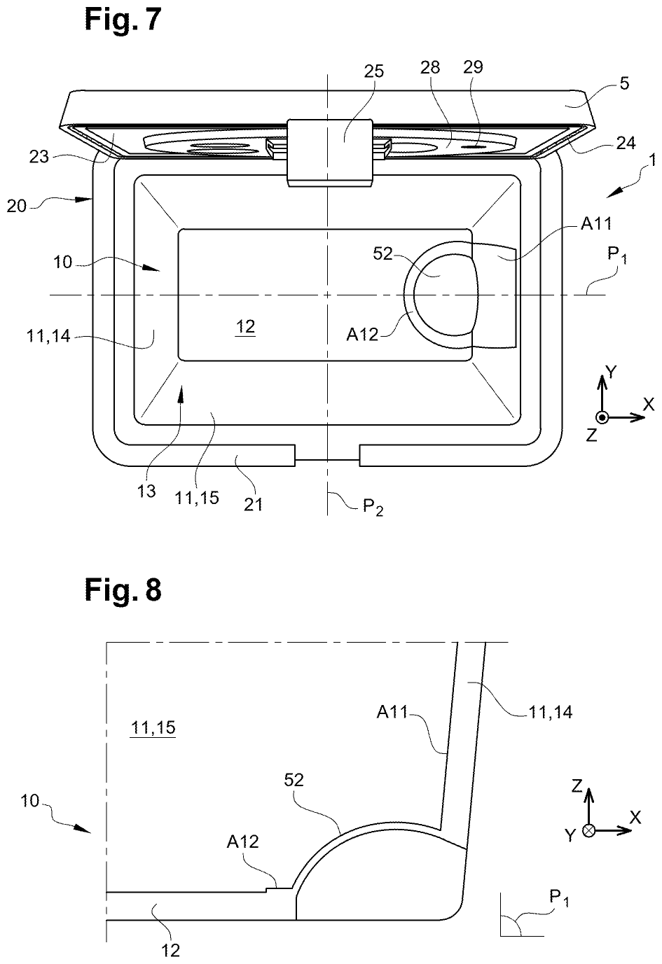

The box 1 has a longitudinal vertical midplane P1 and a transverse vertical midplane P2, as one can see in particular in FIG. 7.

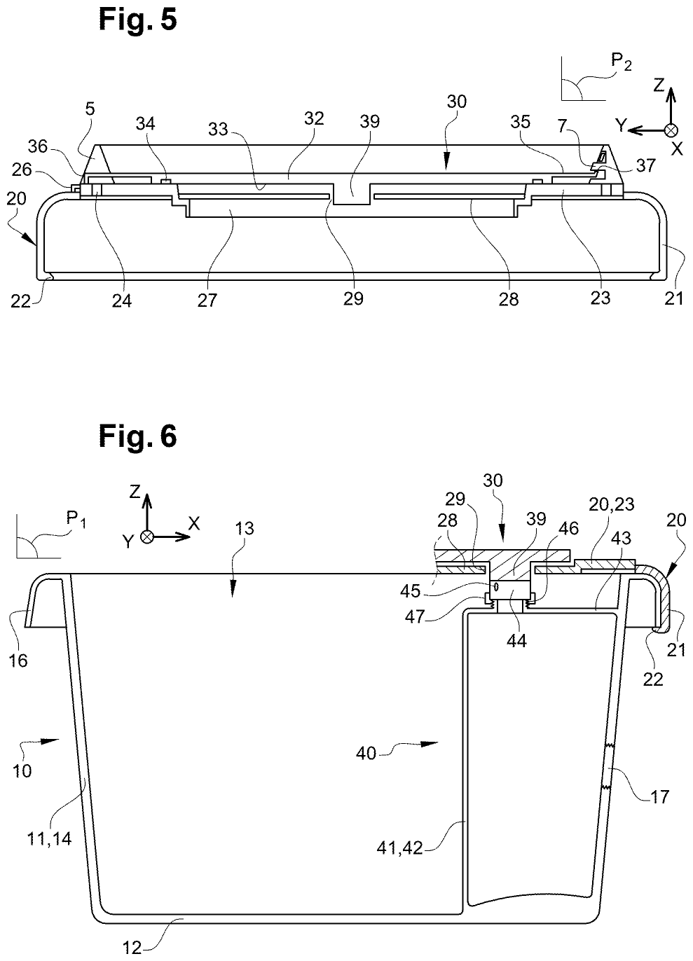

At its upper portion, the peripheral wall 11 may have a flange 16 directed outwards and downwards, which may extend over the entire periphery of the opening 13 (cf. FIG. 5, which is a section according to the plane P2).

The box 1 also includes a plate 20 which is fastened to the receptacle 10. The plate 20 may include a frame 21 which is placed around the opening 13, for example on the flange 16. Preferably, the plate 20 is locked on the receptacle 10, so that the practitioner cannot remove it, only a company specialized in processing the considered wastes being capable of detaching the plate 20 from the receptacle 10 by specific means. To this end, the frame 21 may be provided at its lower portion with an internal bead 22 fitting under the flange 16 of the receptacle 10.

The plate 20 may further include a board 23 which is mounted on the frame 21. When the box 1 is in use, the board 23 substantially covers the opening 13 of the receptacle 10, the board 23 can therefore be substantially rectangular. In this position of use, or closed position of the board 23, the lower face of the board 23 can sealingly rest on the upper edge of the peripheral wall 11 of the receptacle 10, for example on the flange 16. Sealing may be ensured by the interposition of a peripheral seal 24, for example mounted on the board 23. As illustrated in particular in FIG. 7, the board 23 may include a tab 25 able to cooperate with the frame 21, to ensure locking of the board 23 in the closed position on the frame 21.

The board 23 may be pivotably mounted relative to the frame 21, for example via a hinge 26 with an axis parallel to the direction X, located along a large panel 15 of the receptacle 10, opposite the tab 25. The plate 20 may be made in one-piece by molding a plastic material, the hinge 26 being formed of a thin strip connecting the frame 21 and the board 23.

The plate 20 includes at least one aperture 27 for the introduction of wastes into the receptacle 10. The aperture 27 may be disposed substantially centrally with respect to the opening 13 of the receptacle 10; it may be substantially rectangular, according to a possible embodiment.

The aperture 27 may be formed in the board 23. The board 23 may include a depressed area 28, i.e. an area located lower than the rest of the board 23 when the board 23 is in the closed position, in which this aperture 27 is formed. As one can see in FIG. 2, the depressed area 28 may extend beyond the aperture 27, according to the direction X and/or Y. In this instance, it has an oval shape, which is not restrictive.

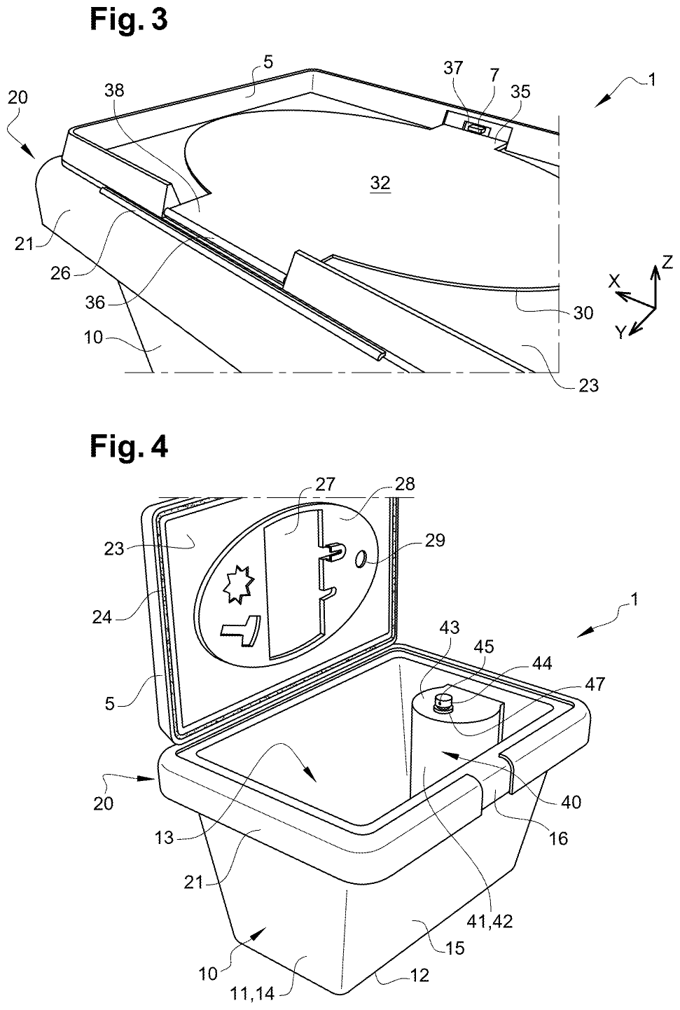

The box 1 further includes a cover 30 which is mounted on the plate 20 movably between an open position (FIGS. 2 and 4), in which the aperture 27 is accessible, and a closed position (FIGS. 1 and 3), in which the cover 30 seals this aperture 27.

The cover 30 may be pivotably mounted relative to the plate 20, for example via a hinge 36 with an axis parallel to the direction X, which may be adjacent to the hinge 26 as one can see in FIG. 3. The cover 30 and the plate 20 may be made in one-piece by molding a plastic material, the hinge 36 being formed of a thin strip connecting the cover 30 and the board 23.

The cover 30 may include a main portion 32, the lower face 33 of which comes into contact with, or immediately proximate to, the upper face of the board 23. The lower face of the cover is also the internal face of the latter, that is to say the face directed towards the inside of the receptacle 10, when the cover 30 is in the closed position.

According to a possible embodiment, and as one can see in FIGS. 1 to 3, the main portion 32 of the cover 30 may have, on the side of its lower face 33, a projecting area which fits into the depressed area 28 of the board 23 in the closed position of the cover 30. Preferably, the projecting area of the cover 30 has a shape complementary to that of the depressed area 28 of the board 23. A sealing gasket 34 may be mounted on the lower face 33 of the cover 30, typically around the projecting area of the main portion 32, to ensure sealing between the plate 20—more specifically the board 23—and the cover 30 in the closed position.

The cover 30 may include an arm 38 connecting the main portion 32 to the hinge 36 and a tab 35 extending from the main portion 32, opposite to the hinge 36. The tab 35 allows locking the cover 30 in the closed position with respect to the plate 20. To this end, the tab 35 may include a hole 37 snapping into a finger 7 of the plate 20. According to one possible embodiment, the board 23 of the plate 20 includes a substantially vertical and peripheral boundary 5, which extends upwards (in the closed position of the board 23), the finger 29 being formed in this boundary 5. The boundary 5 may be interrupted at the level of the arm 38 of the cover 30, as one can see in FIGS. 1 and 3.

Preferably, the cover 30 is locked on the plate 20 in the closed position, and the board 23 is locked on the frame 21 in the closed position, so that unlocking cannot be carried out by the practitioner, but only by a company specialized in the treatment of the considered wastes, by specific means.

In addition, the box 1 includes a cartridge 40, that is to say a small container, which is mounted in the receptacle 10 and which is intended to contain a waste disinfection fluid, generally pressurized. Preferably, the disinfection fluid is a disinfectant product with a broad spectrum, antiviral, antibacterial and fungicidal. It may consist of a gas. The internal volume of the cartridge is determined according to the volume of the receptacle 10, so that the amount of disinfection fluid guarantees effective disinfection.

The cartridge 40 includes a casing 41 and an actuator movable relative to the casing 41 between an inactive position in which the fluid cannot be expelled from the cartridge 40 and an active position in which fluid can be expelled.

As one can see in particular in FIG. 6 (which is a section according to the plane P1) and 11, the casing 41 may include a substantially cylindrical peripheral wall 42, with a substantially vertical axis in the mounted position, and an upper wall 43 having a disk-like general shape.

The actuator may include an endpiece 44 provided with a fluid expulsion hole 45, and function like an aerosol valve. Thus, the endpiece 44 can be mounted in the upper portion of the cartridge 40 movable in vertical translation between a high position—that is to say in the inactive position—and a low position—that is to say in the active position. In concrete terms, the upper wall 43 of the casing 41 may include a collar 46 projecting upwards and provided with an external thread, in which the endpiece 44 is fastened by means of a ring 47 screwed onto the collar 46.

Preferably, the cartridge 40 is disposed in the receptacle 10 proximate to or against the peripheral wall 11 of the receptacle 10. When the aperture 27 is substantially central, this arrangement avoids the cartridge 40 hindering the introduction of the wastes into the receptacle 10. For example, the cartridge 40 may be disposed against one of the small panels 14 of the peripheral wall 11 of the receptacle 10.

According to the present disclosure, the cover 30 includes means for activating the actuator of the cartridge 40, i.e. herein the endpiece 44. The box 1 is configured so that, when the cover 30 is in the open position, the endpiece 44 is in the high position, and so that the switch of the cover 30 into the closed position causes the switch of the endpiece 44 into the low position by the action of the activation means.

To this end, provision may be made for the endpiece 44 to be arranged opposite an orifice 29 of the plate 20, so that the endpiece 44 and the activation means formed on the cover 30 could cooperate. In the embodiment represented as example, the orifice 29 is formed in the depressed area 28 of the board 23, at a distance from the aperture 27.

Furthermore, as one can see in FIGS. 2 and 5, the activation means may comprise a lug 39 which projects substantially orthogonally from the lower face 33 of the casing 30. The lug 39 is arranged on the cover 30 so that, in the closed position of the cover 30, it is located opposite the orifice 29 of the plate 20, as is clearly visible in FIG. 5.

Thus, the orifice 29 of the plate 20 is opposite both the lug 39 of the cover 30 (in the closed position of the cover) and the endpiece 44 of the cartridge 40. Provision may be made for the orifice 29 to have dimensions slightly larger than those of the lug 39 and/or of the endpiece 44, so as to ensure satisfactory guidance and positioning of these two elements relative to one another, to ensure that they will properly come into contact, consequently, that the lug 39 will cause the proper switch of the endpiece 44 into the low position.

It could be provided that, in the inactive position, that is to say in the low position, the endpiece 44 is located beneath the upper face of the plate 20, and even beneath the lower face of the plate 20, more specifically of the board 23. This allows avoiding the endpiece 44 being lowered, and therefore activated, unintentionally. With this configuration, in the closed position of the cover, the lug 39 should be accommodated in the orifice 29 of the plate 20, and even protrude under the board 23, so as to be able to reach the endpiece 44 and actuate it. This is schematically illustrated in FIG. 6.

Thus, the practitioner receives a ready-to-use, empty and disinfected box 1, with the plate 20 fastened on the receptacle 10, the board 23 closed and locked on the frame 21, and the open cover 30. The cartridge 40 contains a suitable amount of disinfection fluid.

Once the box 1 is full of wastes, the practitioner closes the cover 30. This causes bearing of the lug 39 of the cover 30 on the endpiece 44 of the cartridge 40, and the expulsion of the disinfection fluid through the expulsion hole 45. The fluid remains contained in the box 1, since the latter is sealingly closed, via the seals 24 and 34. Thus, the wastes contained in the box 1 are disinfected. In addition, the cover 30 is locked on the plate 20.

The box 1 is handled by a specialized company, which will conduct the operations necessary for recycling the box.

To this end, the assembly comprising the plate 20 and the cover 30 is detached from the receptacle 10, generally by means of a special machine. Then, the wastes contained in the receptacle 10, and which now no longer pose an infectious risk, are emptied. Afterwards, they will be processed as household wastes. The receptacle 10 is then cleaned and disinfected, so as to be able to be reused. Afterwards, the cartridge should be replaced with a cartridge full of disinfection fluid, or the cartridge 40 should be recharged with disinfecting fluid. The assembly comprising the plate 20 and the cover 30 are mounted again on the receptacle 10. Thus, the box 1 is ready to be reused by a practitioner.

According to one embodiment, the cartridge 40 is mounted in the receptacle 10 in a non-removable manner. In order to make the box 1 reusable, it is necessary to be able to recharge the cartridge 40 with disinfecting fluid, and the box 1 therefore includes means 100 for recharging the cartridge 40 with fluid. Preferably, these recharging means 100 are formed on the cartridge 40 and/or on the receptacle 10 and accessible from outside the box 1.

As one can see in FIG. 6, a threaded hole 17 may be formed in a wall of the casing 41 and/or of the receptacle 10 to receive the recharging means 100, these being intended to sealingly cooperate with the head of a fluid injection system (not represented) and set this head in communication with the internal volume of the cartridge 40. The recharging means 100 will be described later on in more details. In the ready-to-use state, the cartridge 40 forms a closed volume intended to contain the disinfection fluid. To solve the problem of molding such a closed volume, in particular when the cartridge is mounted in the receptacle 10 in a non-removable manner, several solutions may be considered. In general, this consists in providing for the casing to form an open volume in the initial state, and for the box to include at least one closure wall which, in the ready-to-use state, cooperates with the casing to form a closed volume adapted to contain the fluid. This closure wall may be formed at least partially by a wall of the receptacle.

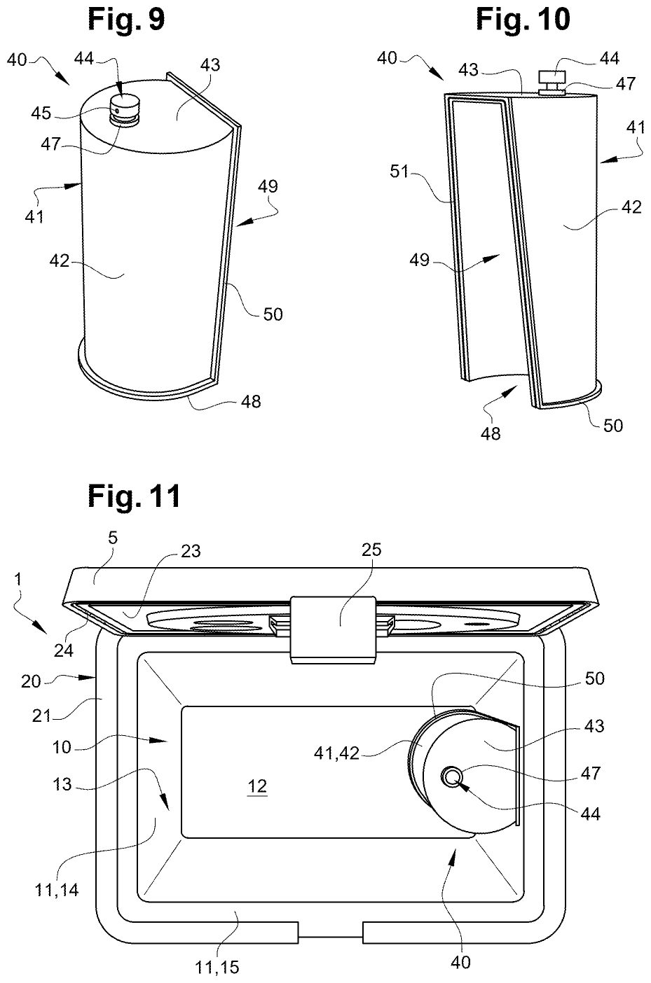

According to one embodiment, illustrated most particularly in FIGS. 9 to 11, the casing 41 is a part distinct from the receptacle 10 in the initial state, that is to say before being mounted in the receptacle 10. For example, the casing 41 is made by molding a plastic material. Once in place in the receptacle 10, the casing 41 and the receptacle 10 may form one single piece.

The casing 41 includes a lower opening 48 and a lateral opening 49 and is sealingly fastened against the internal face of the receptacle 10, so that the bottom wall 12 of the receptacle 10 closes the lower opening 48 and that the peripheral wall 11 of the receptacle 10 closes the lateral opening 49 of the casing 41. The openings 48 and 49 may be in continuity with one another, that is to say globally form one single opening.

For example, the casing 41 may form a cylinder truncated according to a plane substantially parallel to the face of the peripheral wall 11 of the receptacle 10 against which the casing 41 is fastened. This plane may be generally parallel to the axis of the cylinder, or slightly inclined with respect to the latter, typically if the receptacle 10 flares in the direction of its opening 13. In the represented embodiment, the casing 41 is fastened against one of the small panels 14 of the receptacle 10, but other arrangements may be considered.

Thus, an area A12 of the bottom wall 12 of the receptacle 10 forms the lower wall of the cartridge 40, and an area A11 of the peripheral wall 11 of the receptacle 10 forms a portion of the peripheral wall 42 of the casing 41 of the cartridge 40. These areas A12, A11 form a local extra thickness, towards the inside of the receptacle 10, relative to the adjacent areas of the bottom wall 12 and of the peripheral wall 11 of the receptacle 10, respectively, as one can see in FIG. 7. This enables guidance and nesting of the casing 41 against the receptacle 10. The contour of the lower 48 and lateral 49 openings may be provided with a flange 50, for example radial, fitting around and adjacent to the areas A12, A11, to increase the bearing surface between the casing 41 and the internal face of the peripheral wall 11 of the receptacle 10 and enable assembly of the casing 41 to the receptacle 10, for example by heat-welding. A sealing gasket 51 may also be mounted on the face of the flange 50 which will come into contact with the receptacle 10.

Moreover, the area A12 of the bottom wall 12 of the receptacle 10 that forms the bottom of the cartridge 40 advantageously has a portion 52 having a shape cambered towards the inside of the receptacle 10.

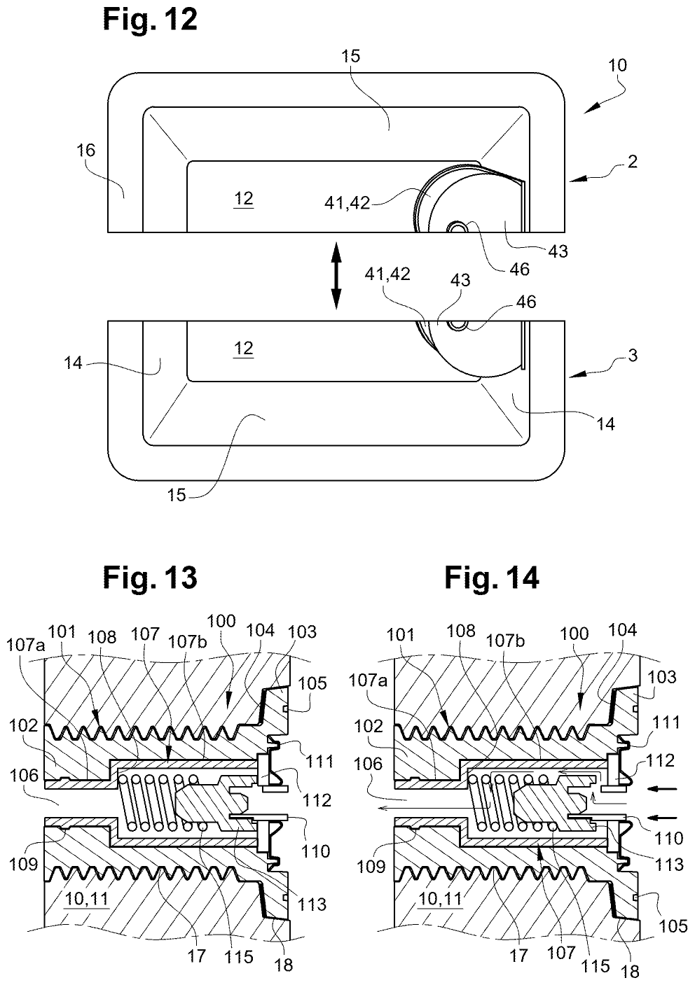

According to another embodiment, illustrated in FIG. 12, the receptacle 10 is formed of several initially distinct portions, herein two portions 2, 3, each corresponding for example to a receptacle 10 half located on one side of the longitudinal vertical midplane P1. Each portion 2, 3 of the receptacle 10 constitutes both a receptacle portion and a casing portion. More specifically, each portion 2, 3 may form one of the large panels 15 of the peripheral wall 11 of the receptacle 10, as well as one half of each of the small panels 14 and one half of the bottom wall 12 and, in addition, one half of the casing 41 (that is to say of the peripheral wall 42 and of the upper wall 43). Each portion 2, 3 may be made in one-piece by molding.

These two portions 2, 3 are sealingly assembled to form the receptacle 10 and the cartridge 40 integrated into the receptacle 10, in the ready-to-use state. For example, the assembly may be made by heat-welding.

With this embodiment, the closure wall of the cartridge, which is open in the initial state, is formed partially by an area of a wall of the receptacle, and partially by a casing half of the cartridge.

Herein again, the area of the bottom wall 12 of the receptacle 10 that forms the bottom of the cartridge 40 preferably has a portion having a shape cambered towards the inside of the receptacle 10.

Reference is now made to FIGS. 13 and 14, which illustrate in more detail an embodiment of the means 100 for recharging the cartridge 40 with fluid.

According to this embodiment, the recharging means 100 include a body 101 having:
a threaded cylindrical portion 102 fitting into the threaded hole 17 which is formed in a wall of the receptacle 10 (such as the peripheral wall 11 in the illustrated example) and communicates with the internal volume of the cartridge 40;
and a collar 103 which can fit into a cavity 18 of said wall of the receptacle 10, surrounding the threaded hole 17 on the external side of the receptacle 10. Preferably, the cavity 18 has a shape corresponding to that of the collar 103, so that in the mounted state, the body 101 is substantially flush with the wall of the receptacle 10 in which it is placed, as one can see in FIGS. 13 and 14. A sealing gasket 104 may be provided between the collar 103 and the cavity 18.

Furthermore, the collar 103 may include a notch 105 into which a tightening tool could be inserted in order to secure mounting of the body 101 in the wall of the receptacle 10.

The body 101 may be made of the same material as the receptacle 10, for example of a plastic material.

The body 101 has an internal channel 106, preferably axial, opening at both ends, i.e. inside and outside the receptacle 10. This channel 106 may have a first portion located on the external side of the receptacle 10 and having a first diameter, and a second portion located on the internal side of the receptacle 10 and having a second diameter smaller than the first diameter. For example, the first portion may extend over about two thirds of the total length of the channel 106.

A sleeve 107 is mounted in the channel 106. The sleeve 107 has an outer shape corresponding to the inner shape of the channel 106. Thus, the sleeve 107 may have a first portion 107a located on the external side of the receptacle 10 and having a first diameter, and a second portion 107b located on the internal side of the receptacle 10 and having a second diameter smaller than the first diameter. Hence, a shoulder 108 is defined between these two portions 107a, 107b.

To ensure the solidity of the mounting, it may be provided that the sleeve 107 includes an outer bead 109, for example in the second portion 107b, inserted into an inner groove of the channel 106. The sleeve may be made of metal, for example of stainless steel.

The sleeve 107 defines a housing in which is received the mechanism that allows:
when it is in the active state, letting the disinfection fluid enter, in order to recharge the cartridge 40 (FIG. 14);
and when it is in the inactive state, ensuring sealing between the inside and the outside of the receptacle 10 (FIG. 13).

In the illustrated embodiment, this mechanism includes an endpiece 110 mounted in the channel 106 in the vicinity of the external end of this channel, by means of a crimping ring 111. An annular seal 112 mounted in the body 101 is disposed around the endpiece 110, and allows ensuring sealing with the head of the injection system during recharging.

In addition, the mechanism includes an opening/closing part 113, which is axially movable between a closed position (illustrated in FIG. 13) and an open position (FIG. 14). A spring 115, bearing on the shoulder 108, pushes the part 113 towards its open position, in which it abuts against the sealing gasket 112 and prevents the passage of disinfection fluid, whether towards the inside or towards the outside of the receptacle 10.

To recharge the cartridge 40, the head of the fluid injection system is introduced into the endpiece 110, which has the effect of displacing the part 113 towards its open position, against the force exerted by the spring 115. Then, as illustrated in FIG. 14, the fluid can circulate in the channel 106 up to the inside of the receptacle 10 and to the internal volume of the cartridge 40.

Once the cartridge 40 is recharged, the head of the injection system is removed from the endpiece 110 and, under the effect of the spring 115, the part 113 returns to its closed position.

Thus, the present disclosure provides a determinant improvement to the prior art, by enabling the reuse of this type of box, and therefore the substantial reduction in the amount of wastes.

It goes without saying that the present disclosure is not limited to the embodiments described hereinabove as examples but that it encompasses all technical equivalents and variants of the described means as well as their combinations.

1. A box for receiving wastes, in particular medical wastes, comprising:
a receptacle including a peripheral wall, a bottom wall, and an opening opposite to the bottom wall;
a plate fastened to the receptacle, substantially covering the opening of the receptacle, and including at least one aperture for introducing wastes into the receptacle;
a cover mounted on the plate movably between an open position, in which the aperture of the plate is accessible, and a closed position, in which the cover seals the aperture;
wherein it further comprises a cartridge adapted to contain a waste disinfection fluid, the cartridge being mounted in the receptacle and including:
a casing;
a fluid expulsion hole;
an actuator movable relative to the casing between an inactive position in which fluid cannot be expelled from the cartridge and an active position in which fluid can be expelled through the expulsion hole;
and wherein the cover comprises means for activating the actuator of the cartridge;
the box being configured so that, when the cover is in the open position, the actuator is in the inactive position, and so that a switch of the cover into the closed position causes a switch of the actuator into the active position by action of the activation means.

2. The box according to claim 1, wherein the activation means comprise a lug projecting substantially orthogonally from an internal face of the cover and wherein the plate comprises an orifice which, when the cover is in the closed position, is located opposite the lug of the cover and the actuator of the cartridge, so that the lug could cooperate with the actuator to make it switch into its active position.

3. The box according to claim 2, wherein, in the closed position of the cover, the lug is at least partially accommodated in the orifice of the plate, the orifice of the plate preferably having dimensions slightly larger than those of the lug.

4. The box according to claim 1, wherein the actuator comprises an endpiece mounted in an upper portion of the cartridge movable in vertical translation, between a high position, corresponding to the inactive position, and a low position, corresponding to the active position, the endpiece being arranged opposite an orifice of the plate so as to be able to cooperate with the activation means formed on the cover.

5. The box according to claim 4, wherein, in the inactive position, the endpiece is located beneath an upper face of the plate.

6. The box according to claim 1, wherein the cartridge is mounted in the receptacle in a non-removable manner, and wherein the box includes means for recharging the cartridge with fluid.

7. The box according to claim 6, wherein the recharging means are formed on the cartridge and/or on the receptacle and are accessible from outside the box.

8. The box according to claim 6, wherein the recharging means include a part which is mounted in a hole of the receptacle, opposite the cartridge, movably between an open position, enabling entry of the waste disinfection fluid into the cartridge, and a closed position ensuring sealing to the fluid between the inside and the outside of the receptacle.

9. The box according to claim 1, wherein the cartridge is disposed in the receptacle proximate to or against the peripheral wall of the receptacle.

10. The box according to claim 1, wherein, in an initial state, a casing forms an open volume, and wherein the box includes at least one closure wall which, in a ready-to-use state, cooperates with the casing to form a closed volume capable of containing the fluid.

11. The box according to claim 10, wherein the closure wall is formed at least partially by a wall of the receptacle.

12. The box according to claim 1, wherein the casing is a part distinct from the receptacle, in an initial state, the casing includes a lower opening and a lateral opening, the casing being sealingly fixed against an internal face of the receptacle, so that the bottom wall of the receptacle forms the bottom of the cartridge and the peripheral wall of the receptacle seals the lateral opening of the casing.

13. The box according to claim 12, wherein an area of the bottom wall of the receptacle that forms the bottom of the cartridge and the area of the peripheral wall of the receptacle that seals the lateral opening of the casing form a local extra thickness, towards the inside of the receptacle, with respect to the adjacent areas of the bottom wall and of the peripheral wall of the receptacle, respectively, enabling guidance and nesting of the casing against the receptacle.

14. The box according to claim 1, wherein it comprises at least two initially distinct portions, each portion constituting a receptacle portion and a casing portion and being preferably made in one-piece by molding, the two portions being sealingly assembled together to form the receptacle and the cartridge integrated into the receptacle.

15. The box according to claim 12, wherein an area of the bottom wall of the receptacle that forms the bottom of the cartridge has a portion having a shape cambered towards the inside of the receptacle.

16. A method for recycling a box, the box configured for receiving wastes, in particular medical wastes, comprising: a receptacle including a peripheral wall, a bottom wall, and an opening opposite to the bottom wall; a plate fastened to the receptacle, substantially covering the opening of the receptacle, and including at least one aperture for introducing wastes into the receptacle; a cover mounted on the plate movably between an open position, in which the aperture of the plate is accessible, and a closed position, in which the cover seals the aperture; wherein the box further comprises a cartridge adapted to contain a waste disinfection fluid, the cartridge being mounted in the receptacle and including: a casing; a fluid expulsion hole; an actuator movable relative to the casing between an inactive position in which fluid cannot be expelled from the cartridge and an active position in which fluid can be expelled through the expulsion hole; and wherein the cover comprises means for activating the actuator of the cartridge; the box being configured so that, when the cover is in the open position, the actuator is in the inactive position, and so that a switch of the cover into the closed position causes a switch of the actuator into the active position by action of the activation means, the cover being in the closed position, the method including the following steps:
detaching an assembly comprising the plate and the cover from the receptacle;
emptying the wastes contained in the receptacle;
cleaning and disinfecting the receptacle;
recharging the cartridge with disinfection fluid, or replacing the cartridge with a cartridge full of disinfection fluid;
mounting the assembly comprising the plate and the cover again on the receptacle.

17. The box according to claim 3, wherein the actuator comprises an endpiece mounted in an upper portion of the cartridge movable in vertical translation, between a high position, corresponding to the inactive position, and a low position, corresponding to the active position, the endpiece being arranged opposite an orifice of the plate so as to be able to cooperate with the activation means formed on the cover.

18. The box according to claim 17, wherein, in the inactive position, the endpiece is located beneath an upper face of the plate.

19. The box according to claim 18, wherein the cartridge is mounted in the receptacle in a non-removable manner, and wherein the box includes means for recharging the cartridge with fluid.

20. The box according to claim 19, wherein the recharging means are formed on the cartridge and/or on the receptacle and are accessible from outside the box.
